(19) United States
(12) Patent Application Publication
(10) Pub. No.: US 2024/0252459 A1
(43) Pub. Date: Aug. 1, 2024

(54) DILTIAZEM HYDROCHLORIDE AND PREGABALIN COMPOSITION, METHOD FOR PREPARING THE SAME, AND USAGE THEREOF

(71) Applicant: HAINAN JIN RUI PHARMACEUTICAL CO., LTD., Haikou City (CN)

(72) Inventors: Yuena Huang, Haikou City (CN); Xiaofang Wang, Haikou City (CN); Jinyu Wang, Haikou City (CN); Shuhua Pan, Haikou City (CN); Shouchun Wang, Haikou City (CN)

(21) Appl. No.: 17/297,269

(22) PCT Filed: May 12, 2021
(86) PCT No.: PCT/CN2021/093185

§ 371 Date: Sep. 14, 2023

(30) Foreign Application Priority Data
Nov. 16, 2020 (CN) 202011277795.2

(51) Int. Cl. A61K 31/197; A61K 9/19; A61K 31/554; A61K 47/26

(52) U.S. Cl. CPC A61K 31/197; A61K 9/19; A61K 31/554; A61K 47/26

(57) ABSTRACT

A diltiazem hydrochloride and pregabalin composition and a preparation method thereof and its application. The composition of the invention is a lyophilizing agent prepared by adding an adequate amount of pregabalin hydrochloride solution, adding excipient and stirring until transparent. The lyophilizing agent will not produce crystallization after dissolution. In addition, by combining proper amount of pregabalin with sufficient amount of diltiazem hydrochloride into an injection, the efficacy of the original diltiazem hydrochloride can be guaranteed, and the adverse reactions such as dyspnea, spasm, skin itching and urticaria can be prevented or reduced.

### FIELD OF THE INVENTION

The present invention relates to pharmaceutical manufacturing technology. Particularly, the invention relates to a diltiazem hydrochloride and pregabalin composition, also relates to a process for the preparation method and an application thereof of this composition.

### BACKGROUND OF THE INVENTION

Diltiazem hydrochloride, the chemical name is cis-(+)-5-[(2-dimethylamino)ethyl]-2-(4-methoxyphenyl)-3-acetoxy-2.3-dihydro-1,5-phenylpropanethiaza-4(5H)-onehydrochloride, molecular formula is c22h26n204s·HCl.

Diltiazem hydrochloride as an oral or injectable agent is effective in dilating the epicardial and subendocardial coronary arteries, relieve spontaneous angina pectoris or angina pectoris caused by coronary artery spasm induced by ergoxin. By slowing your heart rate and lowering your blood pressure, Reduces myocardial oxygen demand, Increased exercise tolerance to relieve exertional angina. It can relax vascular smooth muscle, reduce the resistance of peripheral blood vessels, and reduce blood pressure. Its hypotensive amplitude and the degree of hypertension are concerned, the person with normal blood pressure only makes blood pressure drops slightly. It has a negative inotropic effect and can slow down the conduction of the sinoatrial and atrioventricular nodes.

However, diltiazem hydrochloride may cause a variety of adverse reactions in different populations, which cannot be treated with this drug. The common adverse reactions include pruritus and urticaria. Less common adverse reactions include spasms and dyspnea.

### SUMMARY OF THE INVENTION

To solve the problems in the above technologies, the invention provides a kind of Diltiazem hydrochloride and pregabalin composition, wherein the composition is lyophilized agent which comprising plurality of diltiazem hydrochloride, pregabalin and pharmaceutically acceptable excipient.

Preferably, the dosage of Diltiazem hydrochloride, pregabalin and pharmaceutically acceptable excipient, measured by weight, is 10:70:0.1-0.3.

Preferably, the pharmaceutically acceptable excipient is mannitol.

The invention also provides a process for making the composition of anyone of preceding solutions, said process comprising:
S1) dissolve the Diltiazem hydrochloride with water to prepare solution A;
S2) dissolve the pregabalin with solution A to prepare solution B;
S3) add the pharmaceutically acceptable excipient into solution B to prepare solution C;
S4) add pH regulator into solution C and adjust the pH value between 5.4-5.6 to prepare solution D;
S5) lyophilize the solution D.

Preferably, the dosage of Diltiazem hydrochloride, pregabalin and pharmaceutically acceptable excipient, measured by weight, is 10:60-80:0.1-0.3:1500.

Preferably, the water is sterile water for injection, the pharmacologically acceptable excipient is mannitol.

Preferably, Step S4-1 is also included to conduct a primary sterilizing filtration and a secondary sterilizing filtration of the solution D

Preferably, the time interval between step S1 and the primary sterilizing filtration is less than 4 hours.

The invention also provides the usage of composition as defined in any preceding solutions, wherein the composition is used for the treatment of dilithium hydrochloride receptors which have adverse reactions such as dyspnea, spasm, pruritus and urticaria.

The beneficial effects of the invention are as follows: Of the present invention provides a kind of diltiazem hydrochloride and pregabalin, part of the present invention overcomes the pregabalin as solution, crystal produces in the process of lyophilized after dissolving problems, and by enough with the right amount lyrica made from diltiazem hydrochloride injection, which can guarantee the effectiveness of the original diltiazem hydrochloride. At the same time, it can prevent or reduce its possible adverse reactions such as dyspnea, spasms, skin itching and urticaria.

### THE APPENDED DRAWINGS SHOW

The invention is further explained in detail in the following sections in combination with the attached drawings and specific implementation methods.

FIG. 1 is a block diagram of the preparation process of the invention.

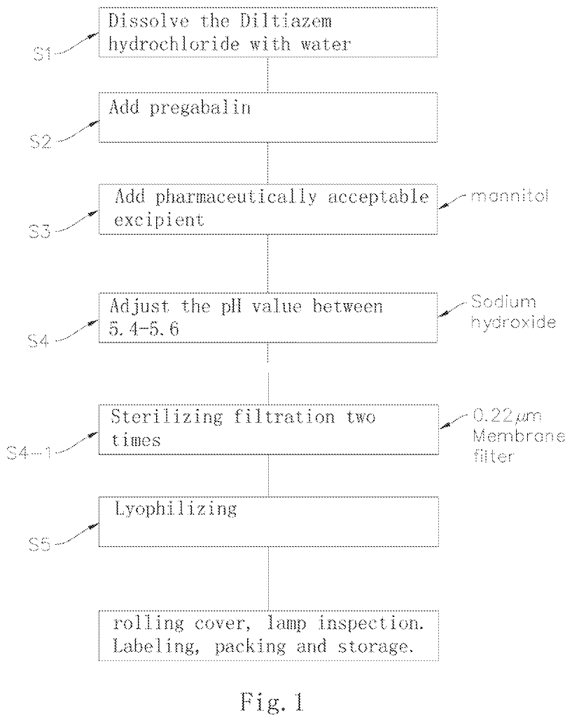

### PREFERABLE EMBODIMENTS

The invention provides a diltiazem hydrochloride and pregabalin composition, which consists of diltiazem hydrochloride (10) by weight of each component, a pharmacologically acceptable excipient (70) and a lyophilized agent of pregabalin (0.1-0.3).

The preparation method is as follows:

1. Dissolve 10 g diltiazem hydrochloride in 1200 ml sterilization water for injection, stir slowly while adding, and prepare 22±2° C. acid solution A.

2. Add 0.1-0.3 g of pregabalin into solution A while stirring slowly to make solution B.

3. Add 70 g of mannitol into solution B while slowly stirring until transparent, and then add to the full amount (1500 ml) with sterilized injection water to prepare solution C.

4. Add 0.5 mol/L sodium hydroxide solution into solution C and adjust pH value to 5.4-5.6 to make solution D.

5. The solution D shall be sterilized and filtered twice:

A sterilization filtration: add the solution D into the pressure filter tank, filter by 0.22 μm membrane, remove impurities and then transport to the liquid storage tank in the liquid acceptance place in the filling room. The preparation of liquid medicine to a sterilization filtration should be completed within 4 hours;

Secondary sterilization and filtration: move the liquid medicine storage tank to the A level laminar flow cover of the filling machine for 0.22 μm secondary terminal sterilization and filtration into the liquid medicine barrel, fill in 1000 bottles of injection agent bottles, half stopper.

5. Lyophilize the solution D;

Pre-lyophilizing: put the injection bottle on the shelf in the lyophilizing cabinet, and put the shelf temperature at 15-20° C., into the box. After the product is put into the box, reduce the shelf temperature to −45±5° C. for no less than 90 minutes. When the product temperature reaches −35° C., keep it warm for 2.5 hours.

A drying: reduce the temperature of the condenser to −50° C., vacuum the system, reduce the vacuum degree to about 10 Pa, shelf temperature for 90 minutes to −5±1° C., until the product ice crystal completely disappeared and continue to keep heat for 3 hours.

Second drying: the shelf slowly (about 60 minutes) to 25±1° C., when the product temperature rises to 20° C. time, keep 4.5 hours, check the vacuum degree no obvious change, end the whole lyophilizing process, vacuum plug, out of the box.

6. rolling cover, lamp inspection.

7. Labeling, packing and storage.

When the combination of diltiazim hydrochloride and pregabalin provided in the invention is used to relieve spontaneous angina pectoris, it can eliminate or reduce dyspnea, spasm, skin itching and urticaria caused by it.

Pregabalin, chemical name (S)-3-aminomethyl-5-methylhexanoic acid, molecular formula is C8H17NO2, is an anti-epileptic drug, clinically mainly for the treatment of postherpetic neuralgia. Because of its hydrophobicity, the drying process in which the lyophilized agent is made results in recrystallization due to the gradual increase in its concentration.

### Example 1

The lyophilized agent made from diltiazepine hydrochloride 10 g, mannitol 70 g and pregabalin 0.1 g by the above method was dissolved in 10 ml pure water at 25° C. and mixed by shock to make an aqueous solution. 0.3 ml of the aqueous solution was placed on a glass slide and dried at 24° C. for 20 hours. No crystallization was observed under a polarizing microscope.

### Example 2

The lyophilized agent made from diltiazepine hydrochloride 10 g, mannitol 70 g and pregabalin 0.2 g by the above method was dissolved in 10 ml pure water at 25° C. and mixed by shock to make an aqueous solution. 0.3 ml of the aqueous solution was placed on a glass slide and dried at 24° C. for 20 hours. No crystallization was observed under a polarizing microscope.

### Example 3

The lyophilized agent made from diltiazim 10 g hydrochloride, mannitol 70 g and pregabalin 0.3 g by the above method was dissolved in 10 ml pure water at 25° C. and mixed by shock to make an aqueous solution. 0.3 ml of the aqueous solution was placed on a glass slide and dried at 24° C. for 20 hours. No crystallization was observed under a polarizing microscope.

### Example 4

The lyophilized agent made from diltiazem hydrochloride 10 g, mannitol 70 g and pregabalin 0.2 g by the above method was dissolved in 10 ml glucose water for 76 patients with pruritus after the use of diltiazem hydrochloride injection, and all patients had the symptoms targeted by diltiazem hydrochloride. Three injections were given at an interval of 3-5 days (according to the corresponding symptoms of the patients), and continuous tracking was recorded until 48 hours after the third injection. The recorded data showed that this product had the corresponding efficacy of diltiazem hydrochloride and could relieve the adverse reactions of skin itching. The specific recorded data were shown in Table 1.

### Example 5

The lyophilized agent made from diltiazem hydrochloride 10 g, mannitol 70 g and pregabalin 0.2 g by the above method was dissolved in 10 ml glucose water for the treatment of 85 patients with pruritus after the use of diltiazem hydrochloride injection, all of whom had the symptoms targeted by diltiazem hydrochloride. Three injections were given at an interval of 3-5 days (according to the corresponding symptoms of the patients), and continuous tracking was recorded until 48 hours after the third injection. The recorded data showed that this product had the corresponding efficacy of diltiazem hydrochloride and could relieve the adverse reactions of dyspnea. The specific recorded data were shown in Table 1.

### Example 6

The lyophilized agent made from diltiazem hydrochloride 10 g, mannitol 70 g and pregabalin 0.2 g by the above method was dissolved in 10 ml glucose water for 64 patients with a record of skin itching after the use of diltiazem hydrochloride injection, and all patients had the symptoms targeted by diltiazem hydrochloride. Three injections were given at intervals of 3-5 days (according to the corresponding symptoms of the patient), and continuous tracking was recorded until 48 hours after the third injection. The recorded data showed that this product had the corresponding efficacy of diltiazem hydrochloride and could relieve the adverse reactions of spasms. The specific recorded data were shown in Table 1.

### Example 7

The lyophilized agent made from diltiazem hydrochloride 10 g, mannitol 70 g and pregabalin 0.2 g by the above method was dissolved in 10 ml glucose water for 91 patients with pruritus after using diltiazem hydrochloride injection, and all patients had the symptoms targeted by diltiazem hydrochloride. Three injections were given at an interval of 3-5 days (according to the corresponding symptoms of the patient), and continuous tracking was recorded until 48 hours after the third injection. The recorded data showed that this product had the corresponding efficacy of diltiazem hydrochloride and could relieve the adverse reactions of urticaria. The specific recorded data were shown in Table 1.

TABLE 1

| | Alle- | | | Adverse | No Adverse | |
|---|---|---|---|---|---|---|
| | viate | sustain | rate | reactions | reactions | rate |
| Example 1 | symptom | | | skin itching | | |
| Injection 1 | 76 | 0 | 100% | 72 | 4 | 94.70% |
| Injection 2 | 75 | 1 | 98.60% | 71 | 5 | 93.40% |
| Injection 3 | 75 | 1 | 98.60% | 73 | 3 | 96% |
| Example 2 | symptom | | | dyspnea | | |
| Injection 1 | 83 | 2 | 97.60% | 81 | 4 | 95.90% |
| Injection 2 | 84 | 1 | 98.80% | 84 | 1 | 98.80% |
| Injection 3 | 84 | 1 | 98.80% | 79 | 6 | 92.90% |
| Example 3 | symptom | | | spasm | | |
| Injection 1 | 64 | 0 | 100% | 64 | 0 | 100% |
| Injection 2 | 63 | 1 | 98.40% | 64 | 0 | 100% |
| Injection 3 | 64 | 0 | 100% | 64 | 0 | 100% |
| Example 4 | symptom | | | urticaria | | |
| Injection 1 | 90 | 1 | 98.90% | 84 | 4 | 92.30% |
| Injection 2 | 89 | 2 | 97.80% | 85 | 5 | 93.40% |
| Injection 3 | 90 | 1 | 98.90% | 87 | 3 | 95.60% |

Symptom records of patients in Implementation Case 1-4 after injection;
Note:
“remission” refers to the obvious effect on the symptoms targeted by diltiazem hydrochloride; “No adverse reaction” refers to the absence or mild symptoms of the corresponding adverse reaction.

In summary, the invention overcomes the problem that pregabalin, as a component of the solution, will crystallize when lyophilized and then dissolved. After an appropriate amount of pregabalin and an adequate amount of diltiazem hydrochloride are combined into an injection, In other words, the efficacy of diltiazem hydrochloride can be guaranteed while the adverse reactions such as dyspnea, cramps, pruritus and urticaria can be prevented or reduced. Therefore, this composition can be used in patients with the adverse reactions described above requiring treatment with diltiazem hydrochloride.

The technical solutions in the Embodiments of the present invention have been described clearly and completely with the drawings of the present invention. Apparently, Embodiments described herein are just a few Embodiments of the present invention. On the basis of Embodiments of the invention, all other related Embodiments made by those skilled in the art without any inventive work belong to the scope of the invention.

1. A diltiazem hydrochloride and pregabalin composition, wherein the composition is lyophilized agent which comprising plurality of diltiazem hydrochloride, pregabalin and pharmaceutically acceptable excipient.

2. The composition of claim 1, wherein the dosage of Diltiazem hydrochloride, pregabalin and pharmaceutically acceptable excipient, measured by weight, is 10:70:0.1-0.3.

3. The composition of claim 2, wherein the pharmaceutically acceptable excipient is mannitol.

4. A process for making the diltiazem hydrochloride and pregabalin composition as claimed in claim 1, wherein said process comprises the following steps:
S1) dissolving the Diltiazem hydrochloride with water to prepare solution A;
S2) dissolving the pregabalin with solution A to prepare solution B;
S3) adding the pharmaceutically acceptable excipient into solution B to prepare solution C;
S4) adding pH regulator into solution C and adjust the pH value between 5.4-5.6 to prepare solution D; and
S5) working to lyophilize the solution D.

5. The process of claim 4, wherein the dosage of Diltiazem hydrochloride, pregabalin and pharmaceutically acceptable excipient, measured by weight, is 10:60-80:0.1-0.3:1500.

6. The process of claim 5, wherein the water is sterile water for injection, the pharmacologically acceptable excipient is mannitol.

7. The process of claim 6, wherein Step S4-1 is also included to conduct a primary sterilizing filtration and a secondary sterilizing filtration of the solution D

8. The process of claim 7, wherein the time interval between step S1 and the primary sterilizing filtration is less than 4 hours.

9. Usage of the diltiazem hydrochloride and pregabalin composition as in claim 1, wherein the composition is used for the treatment of diltithium hydrochloride receptors which have adverse reactions such as dyspnea, spasm, pruritus and urticaria.

10. Usage of the diltiazem hydrochloride and pregabalin composition as claimed in claim 4, wherein the composition is used for the treatment of diltithium hydrochloride receptors which have adverse reactions such as dyspnea, spasm, pruritus and urticaria.

11. A process for making the diltiazem hydrochloride and pregabalin composition as claimed in claim 2, wherein said process comprises the following steps:
S1) dissolving the Diltiazem hydrochloride with water to prepare solution A;
S2) dissolving the pregabalin with solution A to prepare solution B;
S3) adding the pharmaceutically acceptable excipient into solution B to prepare solution C;
S4) adding pH regulator into solution C and adjust the pH value between 5.4-5.6 to prepare solution D; and
S5) working to lyophilize the solution D.

12. A process for making the diltiazem hydrochloride and pregabalin composition as claimed in claim 3, wherein said process comprises the following steps:
S1) dissolving the Diltiazem hydrochloride with water to prepare solution A;
S2) dissolving the pregabalin with solution A to prepare solution B;
S3) adding the pharmaceutically acceptable excipient into solution B to prepare solution C;
S4) adding pH regulator into solution C and adjust the pH value between 5.4-5.6 to prepare solution D; and
S5) working to lyophilize the solution D.

13. Usage of the diltiazem hydrochloride and pregabalin composition as in claim 2, wherein the composition is used for the treatment of diltithium hydrochloride receptors which have adverse reactions such as dyspnea, spasm, pruritus and urticaria.

14. Usage of the diltiazem hydrochloride and pregabalin composition as in claim 3, wherein the composition is used for the treatment of diltithium hydrochloride receptors which have adverse reactions such as dyspnea, spasm, pruritus and urticaria.

15. Usage of the diltiazem hydrochloride and pregabalin composition as claimed in claim 5, wherein the composition is used for the treatment of diltithium hydrochloride receptors which have adverse reactions such as dyspnea, spasm, pruritus and urticaria.

16. Usage of the diltiazem hydrochloride and pregabalin composition as claimed in claim 6, wherein the composition is used for the treatment of diltithium hydrochloride receptors which have adverse reactions such as dyspnea, spasm, pruritus and urticaria.

17. Usage of the diltiazem hydrochloride and pregabalin composition as claimed in claim 7, wherein the composition is used for the treatment of diltithium hydrochloride receptors which have adverse reactions such as dyspnea, spasm, pruritus and urticaria.

18. Usage of the diltiazem hydrochloride and pregabalin composition as claimed in claim 8, wherein the composition is used for the treatment of diltithium hydrochloride receptors which have adverse reactions such as dyspnea, spasm, pruritus and urticaria.
